(19) United States
(12) Patent Application Publication
(10) Pub. No.: US 2006/0195020 A1
(43) Pub. Date: Aug. 31, 2006

(54) Methods, systems, and apparatus for measuring a pulse rate

(76) Inventors: James S. Martin, Atlanta, GA (US); James W. Larsen, Suwanee, GA (US); Peter H. Rogers, Atlanta, GA (US); Michael D. Gray, Atlanta, GA (US); Dan Benardot, Atlanta, GA (US); Alfred Martin, Atlanta, GA (US)

Correspondence Address: JOHN S. PRATT, ESQ;KILPATRICK STOCKTON, LLP, 1100 PEACHTREE STREET, ATLANTA, GA, 30309, US

(21) Appl. No.: 11/266,110

(22) Filed: Nov. 3, 2005

Related U.S. Application Data
(63) Continuation-in-part of application No. 10/903,407, filed on Jul. 30, 2004.
(60) Provisional application No. 60/491,927, filed on Aug. 1, 2003.

(51) Int. Cl. A61B 5/00; A61B 5/02; A61B 5/103; A61B 5/117

(52) U.S. Cl. 600/301; 600/595; 600/500; 128/920

(57) ABSTRACT

The present invention includes methods, systems, and apparatus for measuring a pulse rate. One aspect includes a method for measuring a pulse rate of an individual. The method can include providing at least one sensor adapted to monitor a pulse associated with a user, and further adapted to monitor motion associated with the user. Furthermore, the method can include detecting a pulse associated with a user with the at least one sensor, and detecting motion associated with a user with the at least one sensor. In addition, the method can include generating a signal based at least on the detected pulse associated with the user, and modifying the signal based at least on the motion associated with the user. Moreover, the method can include determining a pulse rate associated with the user based at least on the modified signal.

### RELATED APPLICATIONS

This application is a continuation-in-part application, and claims priority to U.S. Ser. No. 10/903,407, entitled “Methods, Systems, and Apparatus for Monitoring Within-Day Energy balance Deviation,” filed on Jul. 30, 2004, which claims priority to U.S. Ser. No. 60/491,927, entitled “Methods and Devices for Monitoring Within-Day Energy Balance Deviation,” filed on Aug. 1, 2003, the contents of which are hereby incorporated by reference.

### FIELD OF THE INVENTION

The present invention relates to methods, systems, and apparatus for health management monitoring and, more specifically, to health management devices, processes and systems that measure a pulse rate.

### BACKGROUND OF THE INVENTION

Conventional health monitoring devices and methods have relied on a variety of measurements of a person's bodily functions to assess that person's health. Several such devices and methods are widely used for the determination of heart rate. One common automated method is electrocardiography (ECG), which involves the measurement of electrical potentials across the heart. Another common manual technique is palpation of the radial artery at the wrist. Other devices and methods can include both oscillometry and auscultation of the brachial or radial arterial pressure when the artery is partially occluded by an inflatable cuff around the upper arm or forearm. Optical pulse oxymetry is a commonly used non-invasive technique during medical procedures. Wrist wearable devices based on this technique have been developed for sleep monitoring applications. In sleep monitors, the transducers are clamped on the subject's fingers and attached to the wrist-worn device with short cables. A technique that is frequently used on patients with cannulated arteries is invasive arterial tonometry. In this technique a pressure transducer is inserted in the cannula and the arterial blood pressure is recorded in real time. The signal acquired in this way contains more information than would be accessible through conventional non-invasive sphygmomanometry, which determines only two features of the pressure's time waveform. One goal of published research is the determination of the blood pressure's time waveform, however such research has not yet provided reliable health monitoring methods and devices.

Oftentimes a single appendage offers insufficient location options for ECG electrodes (since it is on a single side of the heart) thereby precluding the use of at least one pulse measurement technique. A person's wrist may be too thick for some types of optical absorption measurements. Invasive techniques can require medical supervision and are unlikely to be consumer acceptable. Oscillometric methods may not be suitable for real time monitoring. While some oscillometric wrist-wearable sphygmomanometers can provide somewhat reliable pulse measurements, such devices can require relatively long measurement periods (over 30 seconds for one wrist monitor model tested) and can be easily confounded by an individual's arm motion during measurements.

One type of health monitoring measurement device uses noninvasive tonometry of the radial artery for the measurement of blood pressure. However, measurements using this blood pressure measurement device can be compromised by noise introduced by arm motion of the patient. Furthermore, ongoing development appears to be focused on calibration of the measured pressure while the arm is stationary.

Therefore a need exists for improved methods, systems, and apparatus for health management monitoring. Furthermore, a need exists for health management devices, processes and systems that measure a pulse rate. Moreover, a need exists for devices, systems, and methods for measuring a pulse rate of an individual. Furthermore, a need exists for devices, systems, and methods for measuring a pulse rate of an individual while a portion of the individual's body is in motion.

### SUMMARY OF THE INVENTION

Embodiments of the invention provide some or all of the needs described above. Aspects of the invention provide systems, methods, and apparatuses that measure a pulse rate. One aspect of one embodiment of the invention includes a noninvasive, pressure sensitive device. The device can be incorporated into or with a wrist worn device, such as an article of clothing, a wrist worn device, or an “Energy Watch.” The pressure sensitive device can include one or more sensors capable of detecting motion or movement associated with a user or individual. The pressure sensitive device can also include one or more sensors capable of detecting a pulse associated with the user or individual. Respective signals representing the motion or movement, such as the motional noise, can be subtracted or otherwise processed with signals representing the detected pulse of the user or individual to generate a measure of instantaneous pulse rate associated with the user or individual.

Some aspects of the invention can rely upon a linear-type relationship between an individual's pulse rate and his energy expenditure. In these aspects, continuous pulse monitoring can provide a history of an individual's energy expenditure which can be updated in real time. Such monitoring can serve as a health-related aid including, but not limited to, a weight loss regimen or a physical training regimen for the individual. One embodiment of the invention, such as a wrist-worn watch-type device, can determine energy expenditure in an ergonomic and compact device which can continuously measure an individual's pulse at the wrist where the device is worn rather than by means of additional apparatuses such as chest straps, which are commonly used for pulse monitoring during exercise. Such a device can include transduction and processing capabilities for real-time pulse measurement and energy expenditure estimation. The device can include wrist-based sensors and algorithms to detect and measure the individual's pulse when the individual is at rest or during periods of physical activity.

One aspect according to one embodiment of the invention includes a method for measuring a pulse rate of an individual. The method can include providing at least one sensor adapted to monitor a pulse associated with a user, and further adapted to monitor motion associated with the user. Furthermore, the method can include detecting a pulse associated with a user with the at least one sensor, and detecting motion associated with a user with the at least one sensor. In addition, the method can include generating a signal based at least on the detected pulse associated with the user, and modifying the signal based at least on the motion associated with the user. Moreover, the method can include determining a pulse rate associated with the user based at least on the modified signal.

According to another aspect of the invention, the method can also include calculating an energy balance based at least on the pulse rate, and outputting an energy balance calculation to the user.

According to yet another aspect of the invention, the method can also include transmitting the pulse rate to a processing device adapted to store the pulse rate.

According to another aspect of the invention, the at least one sensor can be mounted to the user on at least one of the following locations: arm, leg, head, neck, chest, calf, ankle, wrist, finger, hand, foot, toe, or a body part.

According to another aspect of the invention, the at least one sensor can be mounted to at least one of the following: a wrist-worn device, a casing, a patch, a band, or an article of clothing.

According to another aspect of the invention, the at least one sensor comprises at least one of the following: a piezoelectric sensor, a force transducer, a pressure transducer, an electret foam sensor, a pressure sensor, a non-invasive tonometric sensor, a motion sensor, an accelerometer, or an array of pressure sensors and motion sensors.

Another aspect according to one embodiment of the invention includes an apparatus for measuring pulse rate of an individual. The apparatus can include at least one sensor and a processor. The at least one sensor is adapted to detect a pulse associated with a user with the at least one sensor, detect motion associated with a user with the at least one sensor, and generate a signal based at least on the detected pulse associated with the user. Furthermore, the processor is adapted to receive the signal from the at least one sensor, modify the signal based on at least the motion associated with the user, and determine a pulse rate associated with the user based at least on the modified signal.

According to another aspect of the invention, the apparatus can include an output device adapted to display the pulse rate.

According to another aspect of the invention, the processor is further adapted to calculate an energy balance based at least on the pulse rate, and output an energy balance calculation to the user.

According to another aspect of the invention, the processor is further adapted to transmit the pulse rate to a processing device adapted to store the pulse rate.

According to another aspect of the invention, the at least one sensor can be mounted to the user on at least one of the following locations: arm, leg, head, neck, chest, calf, ankle, wrist, finger, hand, foot, toe, or a body part.

Another aspect according to one embodiment of the invention includes a system for measuring pulse rate of an individual and determining an energy expenditure of the individual while the individual is in motion. The system can include at least one sensor array and a processor. The at least one sensor array can be capable of detecting a pulse associated with a user with the at least one sensor array, detecting motion associated with a user with the at least one sensor array, and generating a signal based at least on the detected pulse associated with the user. Furthermore, the processor is capable of receiving the signal from the at least one sensor array, modifying the signal based on at least the motion associated with the user, and determining a pulse rate associated with the user based at least on the modified signal. The processor is further capable of calculating an energy balance based at least on the pulse rate, and outputting an energy balance calculation to the user.

According to another aspect of the invention, the system can include an output device capable of displaying the pulse rate.

According to another aspect of the invention, the processor is further capable of transmitting the pulse rate and energy balance to a data storage device capable of storing the pulse rate and energy balance.

According to another aspect of the invention, the at least one sensor array can be mounted to the user on at least one of the following locations: arm, leg, head, neck, chest, calf, ankle, wrist, finger, hand, foot, toe, or a body part.

According to another aspect of the invention, the at least one sensor array comprises at least one pressure sensor and at least one motion sensor.

Objects, features and advantages of various systems, methods, and apparatuses according to various embodiments of the invention include:

(1) providing systems, methods, and apparatuses for health management monitoring;

(2) providing systems, methods, and apparatuses for measuring a pulse;

(3) providing systems, methods, and apparatuses for measuring a pulse rate of an individual; and

(4) providing systems, methods, and apparatuses for measuring a pulse rate of an individual while a portion of the individual's body is in motion; and

(5) providing systems, methods, and apparatuses for measuring a pulse rate of an individual and determining the individual's energy expenditure while the individual's body is in motion.

Other objects, features and advantages of various aspects and embodiments of systems, methods, and apparatuses according to the invention are apparent from the other parts of this document.

### BRIEF DESCRIPTION OF THE DRAWINGS

FIG. 1 illustrates an environment for an embodiment of an apparatus in accordance with the invention.

FIG. 2 illustrates an overhead view of the apparatus shown in FIG. 1.

FIG. 3 illustrates an underside view of the apparatus shown in FIG. 1.

FIG. 4 illustrates a side view of the apparatus shown in FIG. 1.

FIG. 5 illustrates a schematic view of an embodiment of an apparatus in accordance with the invention.

FIG. 6 illustrates an example sensor array for an embodiment of an apparatus in accordance with the invention.

FIG. 7 illustrates two examples of a pressure sensor for embodiments of an apparatus in accordance with the invention.

FIG. 8 illustrates comparative examples of pulse time histories measured with an ECG and a pressure sensor for an embodiment of an apparatus in accordance with the invention.

FIG. 9 illustrates comparative examples of pulse signal spectrograms measured with an ECG and a pressure sensor for an embodiment of an apparatus in accordance with the invention.

FIG. 10 illustrates comparative examples of signals measured with an ECG, and a pressure sensor and a motion sensor for an embodiment of an apparatus in accordance with the invention.

FIG. 11 illustrates comparative examples of signal spectrograms measured with an ECG, and a pressure sensor and a motion sensor for an embodiment of an apparatus in accordance with the invention.

FIG. 12 illustrates an example of a corrected signal spectrogram measured with a pressure sensor and a motion sensor for an embodiment of an apparatus in accordance with the invention.

FIG. 13 illustrates comparative examples of pulse rate measurements for an embodiment of an apparatus in accordance with the invention.

FIG. 14 illustrates an embodiment of a method in accordance with the invention.

FIG. 15 illustrates another embodiment of a method in accordance with the invention.

### DETAILED DESCRIPTION

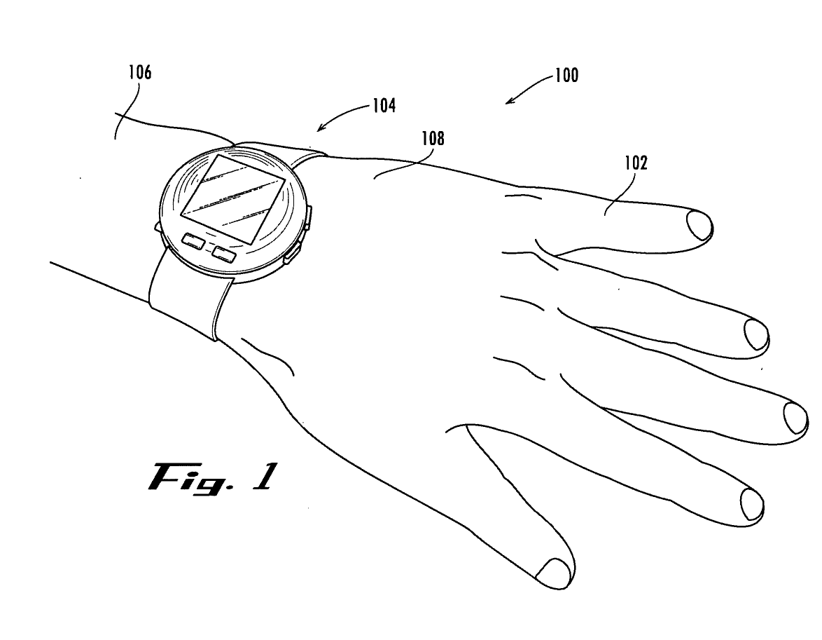

FIGS. 1-4 illustrate an embodiment of an apparatus in accordance with the invention. FIG. 1 illustrates an environment for an embodiment of an apparatus in accordance with the invention. The environment 100 shown in FIG. 1 is an individual 102 wearing an apparatus such as a wrist-worn device 104 for measuring pulse rate. The wrist worn device 104 shown in FIG. 1 can include one or more sensors capable of measuring the pulse rate of the individual 102 while a portion of the individual's body is in motion, such as the individual's arm 106 or wrist 108. In general, a user or individual 102 may swing either or both arms while the individual is walking, jogging, or running. The one or more sensors associated with the wrist worn device 104 can detect the individual's pulse and motion associated with the individual, such as the movement of the individual's arm 106 or wrist 108. The one or more sensors associated with the wrist worn device 104 can generate a signal associated with the detected pulse, and modify the signal based in part on the motion or movement of the individual's arm 106 or wrist 108. A pulse rate of the individual 102 can then be determined based at least in part on the modified signal.

In other embodiments, the apparatus can include, but is not limited to, an article of clothing such as a watch, a hat, a shirt, pants, or a shoe. Furthermore, in other embodiments, one or more sensors can be mounted to the user on at least one location including, but not limited to, an arm, leg, head, neck, chest, calf, ankle, wrist, finger, hand, foot, toe, a body part, or any combination of locations.

In one embodiment, an apparatus such as the wrist worn device 104 can function with, or otherwise incorporate some or all functionality associated with, a “Health Watch,” as disclosed by “Methods, Systems, and Apparatus for Monitoring Within-Day Energy Balance Deviation,” U.S. Ser. No. 10/903,407, filed Jul. 30, 2004; and “Method and Device for Monitoring Within-Day Energy Balance Deviation,” U.S. Ser. No. 60/491,927, filed Aug. 1, 2003; wherein the contents of both applications are incorporated by reference. For example, an apparatus such as the wrist worn device 104 shown in FIG. 1 can provide information related to a person's energy expenditure, such as a pulse rate over a period of time. The pulse rate can be used as an input to an algorithm capable of calculating an energy balance function based in part on an energy expenditure and energy intake over a period of time. The wrist worn device 104 could then determine, output, or otherwise display information associated with the energy balance function.

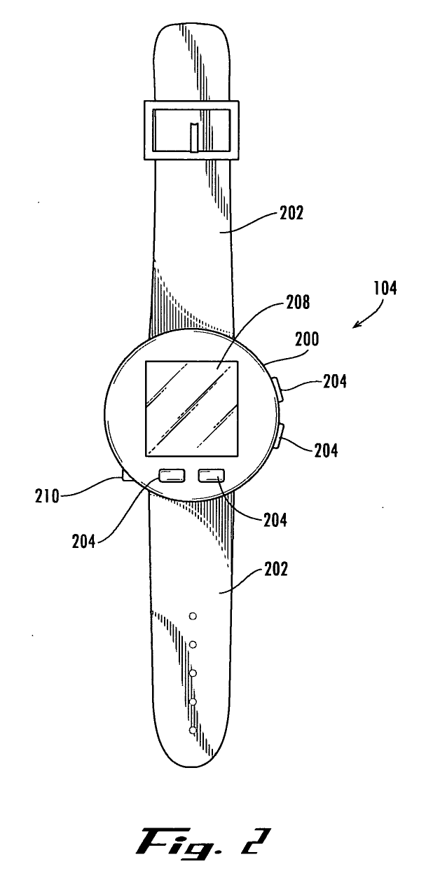

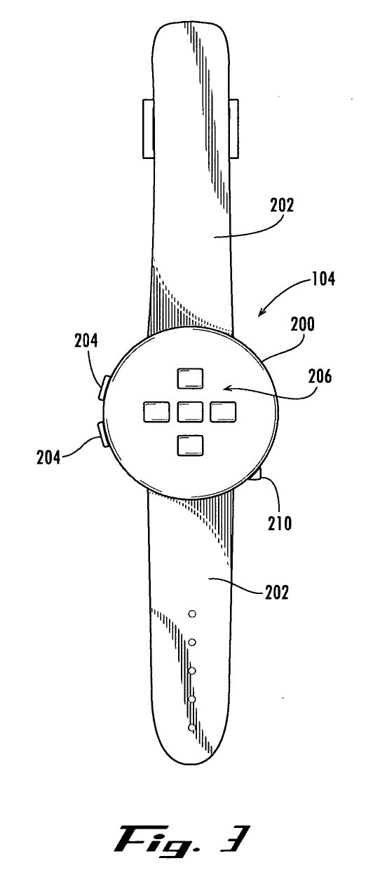

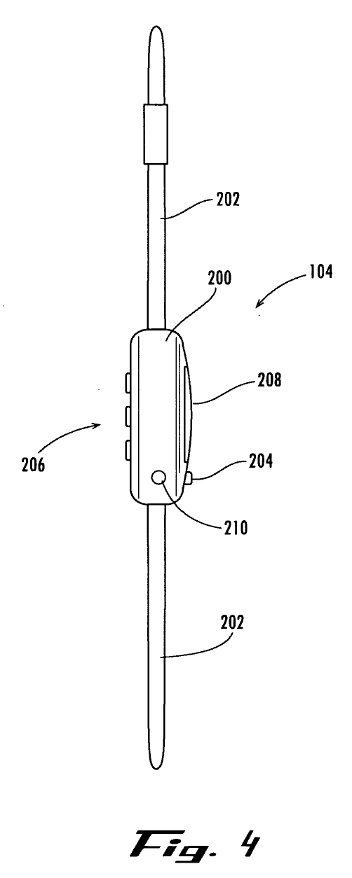

FIG. 2 illustrates an overhead view, FIG. 3 illustrates an underside view, and FIG. 4 illustrates a side view of the apparatus shown in FIG. 1. The apparatus, a wrist-worn device 104, shown in FIGS. 1-4 includes a casing 200, mounting strap 202, one or more input buttons 204, sensor array 206, output display 208, and an output port 210. Other embodiments can include fewer or greater numbers of components, and alternative arrangements or configurations of components, in accordance with the invention.

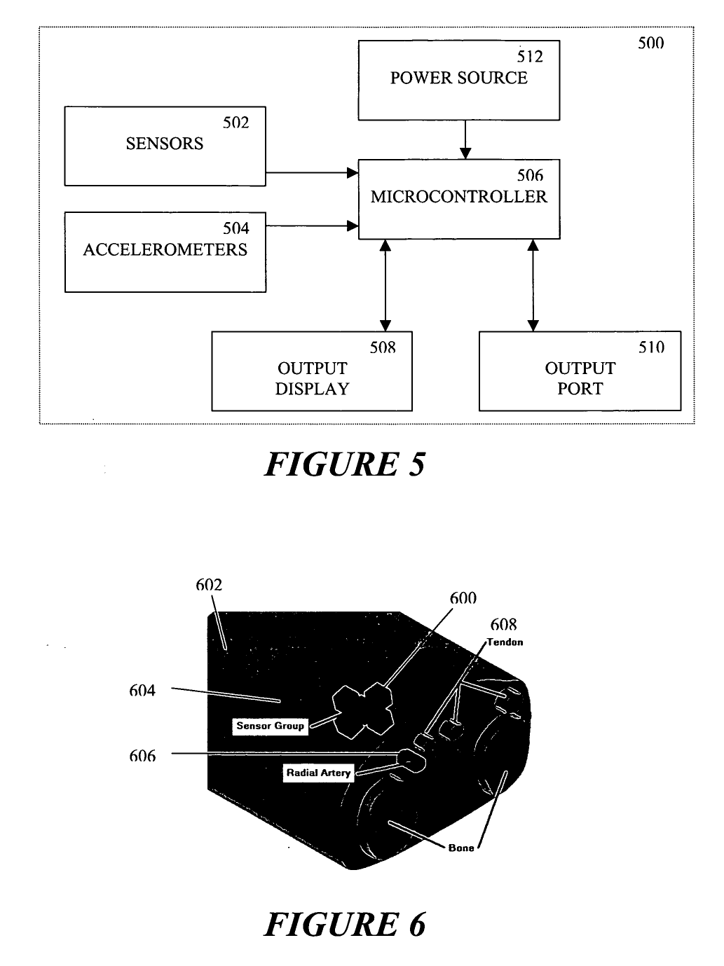

The casing 200 shown in FIGS. 2-4 can be adapted to house, contain, or otherwise mount one or more electronic components capable of processing inputs received or detected from a user or individual, such as one or more signals associated with a detected pulse rate and motion associated with a user or individual 102. The electronic components can be further capable of modifying a signal associated with the detected pulse rate based at least in part on the motion associated with the user or individual 102 shown in FIG. 1, and determining a pulse rate associated with the user or individual based at least in part on the modified signal. One example of an embodiment of electronic components for processing inputs received or detected from an individual is shown and described in FIG. 5.

In at least one embodiment, a casing 200 can comprise a surface with one or more electronic components mounted to the surface. The electronic components can be capable of processing inputs received or detected from a user or individual, such as one or more signals associated with a detected pulse rate and motion associated with a user or individual 102 shown in FIG. 1. Moreover, the electronic components can be further capable of modifying a signal associated with the detected pulse rate based at least in part on the motion associated with the user or individual 102, and determining a pulse rate associated with the user or individual based at least in part on the modified signal.

In another embodiment, a casing 200 can include a combination of electronic components inside the casing 200 as well as mounted to an external surface associated with the casing 200.

As shown in FIGS. 2-4, the mounting strap 202 can mount the casing 204 directly to or adjacent to a user or individual 102, such a user's arm 106 or wrist 108 shown in FIG. 1. The mounting strap 202 can be a strap-type device, or can be any other type of device capable of mounting the casing 204 directly, adjacent, or proximate to a portion of a user's body. In one embodiment, an adhesive, gel, or other material can be used to mount the casing 204 directly, adjacent, or proximate to a portion of a user's body. In another embodiment, In any instance, the casing 204 can be mounted directly to, adjacent to, or proximate to a portion of a user's body.

In one embodiment, a mounting strap 202 can be capable of providing a predetermined amount of static pressure for the sensor array 206 to ensure a suitable mounting or contact of the sensor array 206 directly to, adjacent, or proximate to a user or individual's skin surface. The predetermined static pressure can also account for, or otherwise be suitable with, a user or individual's comfort, mobility and/or blood circulation.

The one or more input buttons 204 shown in FIGS. 2-4 can be adapted to receive an input or command from a user or individual 102. Associated inputs or commands received from the individual 102 via the input buttons 204 can be processed by the wrist worn device 104 as needed. In one embodiment, a menu-driven program or set of instructions associated with a processor can prompt or otherwise receive input and commands from a user or individual 102. The number, arrangement, and configuration of the input buttons 204 shown in FIGS. 2-4 is by way of example only. Other embodiments can include greater or fewer input buttons, alternative arrangements and configurations with respect to the casing 200. Other examples of input buttons are described with respect to FIG. 5.

In at least one embodiment, input buttons can be replaced by a receiver or microphone adapted to receive a speech or voice command or input from a user or individual 102. In another embodiment, a combination of input and commands can be received via one or more input buttons and a receiver or microphone adapted to receive a speech or voice command or input from a user or individual 102.

The sensor array 206 shown in FIGS. 2-4 can be adapted to detect a pulse rate of a user or individual 102, and can be further adapted to detect a motion associated with the user or individual 102. In the example shown, a sensor array 206 can include one or more sensors capable of being in direct contact with a portion of a user's body, such as an arm 106 or wrist 108. One example of a sensor array in accordance with an embodiment of the invention is shown as 600 described below with respect to FIG. 6. In other embodiments, a sensor array 206 can include one or more sensors capable of being proximate or adjacent to a portion of a user's body, or may include a combination of sensors in direct contact with as well as proximate or adjacent to a user's body. In any instance, the sensor array 206 can generate one or more signals associated with the detected pulse rate and/or detected motion associated with the user or individual 102. The one or more signals can then be processed by the wrist worn device 104 as needed.

Other embodiments of a sensor array 206 can include greater or fewer sensors, and alternative arrangements and configurations of sensors or sensor arrays with respect to the casing 200. In one embodiment, a sensor array 206 can be mounted in a mounting strap, such as 202, or in both the casing 200 and the mounting strap 202. In another embodiment, a sensor array 206 can be configured adjacent to or proximate to a user or individual's radial artery. Other examples of a sensor array are described with respect to FIG. 5.

The output display 208 shown in FIGS. 2-4 can be adapted to display or otherwise output text or at least one indicator to a user or individual 102. An output display can include, but is not limited to, a LED or a LCD. Other embodiments of an output display 208 can include a loudspeaker or audio device adapted to provide an output to a user or individual 102, such as a voice or audible indicator. Another embodiment of an output display 208 can include a device capable of providing a tactile indication such as a vibration. Other examples of an output display are described with respect to FIG. 5.

The output port 210 shown in FIGS. 2-4 can be adapted to output at least one signal to a remote device, such as a processing device or a data storage device. An output port can include, but is not limited to, a serial port, a USB port, or any other type of input/output port. Other examples of an output port are described with respect to FIG. 5.

FIG. 5 illustrates a schematic view of example components associated with an apparatus in accordance with the invention. In the example shown in FIG. 5, an apparatus 500 can include one or more sensors 502, one or more motion sensors such as accelerometers 504, at least one microcontroller 506, an output display 508, an output port 510, and a power source 512. Each of the components and their associated functionality is described below. Other embodiments of the apparatus can include some or all of these components in accordance with the invention.

The apparatus 500 shown in FIG. 5 can be mounted to a user or individual in a variety of ways, such as incorporated in an article of clothing. Examples of an article of clothing can include, but are not limited to, a wrist worn device, a watch, a shirt, pants, a patch, a strap, a button, or a hat. The apparatus 500 can also be mounted directly, proximate, or adjacent to a portion of user or individual's body by way of an adhesive, gel, or other material capable of facilitating substantially close contact between an external surface the apparatus and the skin of the user or individual.

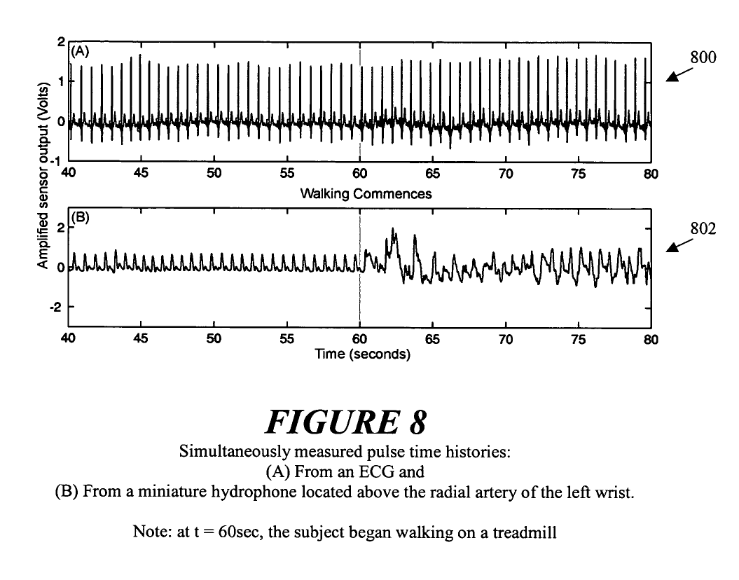

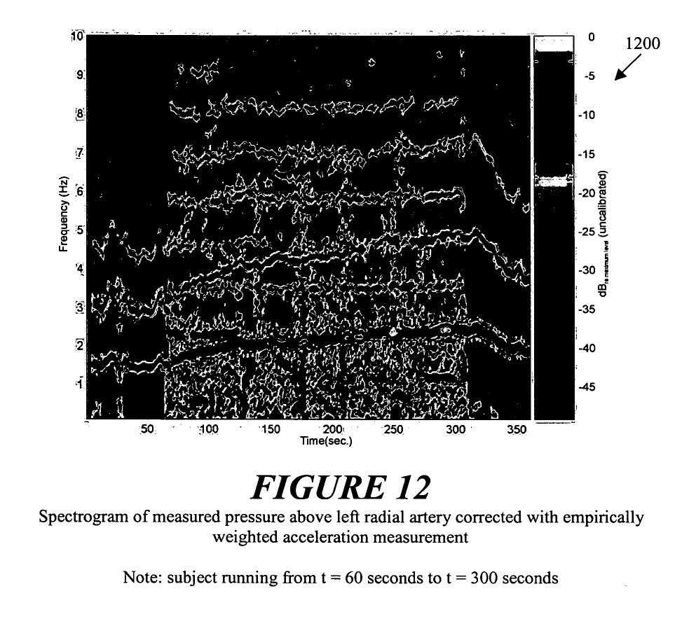

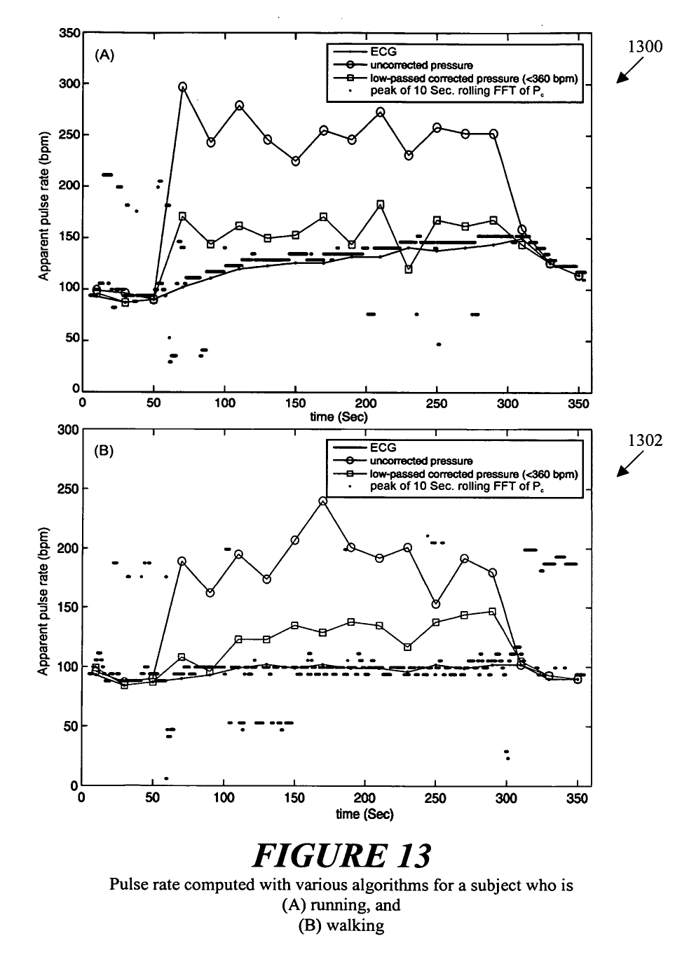

When the apparatus 500 is worn by a user or individual, one or more sensors 502 can detect a pulse associated with a user, and one or more motion sensors, such as accelerometers 504, can detect motion or movement associated with the user. The sensors 502 and motion sensors such as accelerometers 504, for instance, can each generate signals based on the respective detected pulse and detected motion or movement of the user. Examples of signals associated with the sensors and motion sensors are illustrated in FIGS. 8-11. The respective signals can be then be transmitted to the microcontroller 506 or other processing device. The microcontroller 506 can process the respective signals, and modify one or more signals associated with the pulse based at least on the motion or movement associated with the user. One or more modified signals can be generated by the microcontroller 506, and a pulse rate associated with the user can be determined based at least on the modified signals. An example of modified signals are illustrated in FIG. 12. The microcontroller 506 can then output the pulse rate to the output display 508 for viewing or analysis by the user or individual. An example of a pulse rate prior to and after signal processing is illustrated in FIG. 13. In some instances, signals and/or a pulse rate can be transmitted via the output port 510 to a remote device such as a processing device or a data storage device. In other instances, signals and/or a pulse rate can be transmitted via the output port 510 from a processing device or a data storage device to the microcontroller 506. The power source 512 can provide power or suitable current to each of the sensors 502, accelerometers 504 or motion sensors, microcontroller 506, output display 508, and output port 510 as needed.

The sensor 502 shown in FIG. 5 can include one or more sensors capable of detecting a pulse associated with a user or individual. In one example, at least one sensor 502 such as a pressure sensor can be configured with a wrist worn device, such as a watch, that can be positioned directly, adjacent, or proximate to a user or individual's arm or wrist, and proximate or adjacent to the radial artery of the user or individual. For example, in one embodiment, the sensor 502 can be located adjacent to, or proximate to, a user or individual's radial artery. Various embodiments can incorporate a sensor 502 in an apparatus 500, wherein the sensor 502 can be positioned on any portion of a user or individual's wrist or arm, such as the upper or top portion of a user's wrist, a lateral side of a user's wrist, or the lower or underside of a user's wrist. One example of a suitable pressure sensor is a customized, piezoelectric foam sensor. In one embodiment, one or more sensors 502 can include one or more high impedance preamplifiers and one or more analog to digital converters (ADCs). The high impedance preamplifiers are capable of adjusting the signal level of the sensors 502 to a suitable level for the ADCs to convert one or more output signals from the sensors 502 to a digital format suitable for input to a microcontroller, such as 506. For example, relatively low power pressure sensors in 1, 2, or 4 operational amplifier modules can be utilized in accordance with the invention.

In one embodiment, a sensor 502 can include a pressure sensor or transducer capable of measuring relatively low frequencies associated with a pulse. In the instance of measuring a pulse associated with a user or individual, an upper range of an expected pulse rate can be approximately 220 beats per minute (BPM), which equates to approximately 3.7 Hz. Particular overtones can be observed in a tonometric signal associated with a pulse rate acquired above a radial artery of a user or individual. An expected frequency range associated with such a signal can be approximately <20 Hz.

In another embodiment, a sensor can include a PVDF (Polyvinylidene Fluoride) film or similar piezoelectric material. Such embodiments using a PVDF film or similar piezoelectric material can minimize weight associated with the sensor and can also provide a variety of geometries for configuring or otherwise shaping a sensor. In one example, a PVDF film can have a 2 mil thickness such as a film distributed by Images SI, Inc. of Staten Island, N.Y. (United States). A sensor constructed with such film can be sensitive to both deliberate and arbitrary arm and wrist motions. In addition, the film can provide relatively high mechanical compliance as well as wearer comfort and relatively close fit to a user or individual's arm or wrist.

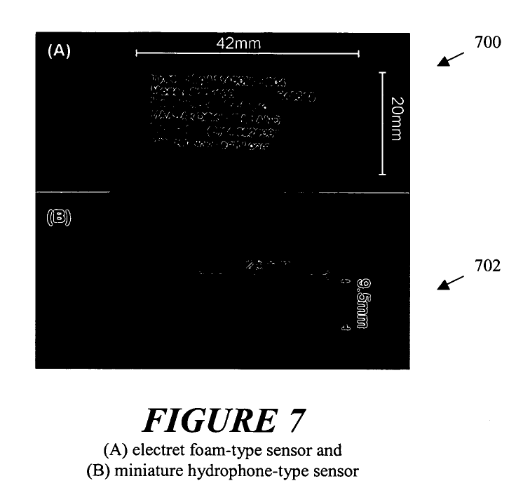

In yet another embodiment, a sensor can include a miniature hydrophone-type sensor. This type of sensor can include a capped cylinder of lead-zirconate-titanate (PZT) piezoelectric ceramic measuring approximately 9.5 mm in diameter and 25 mm in length. Similar to other sensors in accordance with embodiments of the invention, the sensor can be located over the radial artery of a user or individual's arm or wrist to detect a pulse associated with the user or individual. An example of this type of sensor is shown as 702 in the lower portion of FIG. 7.

In yet another embodiment, a sensor can include a model S20X100, electret foam-type sensor distributed by Emfit Ltd. of Finland. This type of sensor can include a relatively thin piezoelectric foam with a rectangular sensitive area measuring approximately 20 mm by 42 mm. Similar to other sensors in accordance with embodiments of the invention, the sensor can be located over the radial artery of a user or individual's arm or wrist to detect a pulse associated with the user or individual. This embodiment can also include an internal charge amplifier to provide suitable signal-to-noise performance. Additional signal enhancing performance may be obtained by selecting various dimensions for the film sensor to further restrict its sensing area to the immediate vicinity of the user or individual's radial artery. In one embodiment of this type of film sensor, a laminate of Sorbothane® rubber and phenolic can be utilized with the sensor to further reduce its sensitivity to flexure. An example of this type of sensor is shown as 700 in the upper portion of FIG. 7.

A motion sensor, such as the accelerometer 504 shown in FIG. 5, can include at least one sensor capable of detecting a movement or motion associated with a user or individual, such a movement of a user's arm or wrist. In one example, a motion sensor can include a preamplifier and a voltage reference device. One example of a suitable motion sensor is an ADXL32x series, iMEMS-type (integrated micro electro mechanical system), dual axis accelerometer distributed by Analog Devices, Inc. of Norwood, Mass. (United States). This type of accelerometer is approximately 0.160 by 0.160 by 0.060 inches (4 mm by 4 mm by 1.45 mm) in size and requires approximately 0.5 mA of current. Another example of a suitable motion sensor is a model W356AI2 miniature triaxial ICP® accelerometer distributed by PCB Piezoelectronics of Depew, N.Y. (United States).

In one embodiment, one or more preamplifiers, if needed, can be utilized in conjunction with a motion sensor in accordance with the invention. An example of a suitable preamplifier is a series 560-type preamp distributed by Stanford Research Systems, Inc. of Sunnyvale, Calif. (United States). Another example of a suitable preamplifier is a monolithic IC opamp such as an OP27 or 37. In another embodiment, a suitable motion sensor can operate at a sufficient output level without a preamplifier.

In at least one embodiment, a voltage reference device can be utilized in conjunction with a motion sensor in accordance with the invention. In some instances, supply voltage for a motion sensor may be sufficiently stable to obviate the need for the use of any voltage reference device with the motion sensor. An example of a suitable voltage reference device is distributed by Zetex PLC of the United Kingdom.

An example of a sensor 502 and motion sensor, such as an accelerometer 504, configured as a sensor array is shown in FIG. 6. In FIG. 6, a sensor array 600 can be positioned directly, adjacent, or proximate to a user or individual's arm 602 or wrist 604, and proximate or adjacent to the radial artery 606 of the user or individual 606. The sensor array 600 shown in FIG. 6 can include one or more sensors capable of detecting a pulse from the radial artery 606, and can further include one or more motion sensors capable of detecting a movement or motion associated with the user or individual, such a movement of a user's arm 602 or wrist 604. In at least one embodiment, a sensor 502 and a motion sensor, such as an accelerometer 504, can be located in an apparatus 500 capable of being positioned on an upper or top side of a user's wrist, a lateral side of a user's wrist, or a lower or under side of a user's wrist. In some embodiments, sensors in a sensor array, such as 600, may overlap one or more wrist tendons 608 when the sensors are positioned to measure signals from the radial artery. In some instances, sensors in a sensor array, or the array configuration itself, can be narrower in shape to reduce any tendon motion sensitivity, while increasing detected pulse signals. To further suppress tendon-related or other similar noise, the sensors in a sensor array can be further subdivided as necessary, from 4 shown in FIG. 6 to a greater or lesser number of sensors, into an array of smaller, independent sensors. In this manner, sensitivity to bending and/or wrinkling along the length of a user's arm 602 or wrist 604 can be reduced. Furthermore, in some embodiments, suppression of residual noise may also occur through associated differential processing of the respective sensor signals. Other embodiments of a sensor array for an apparatus in accordance with the invention can have alternative arrangements and/or configurations.

The microcontroller 506 shown in FIG. 5 can include at least one microcontroller or other suitable processing device capable of providing centralized data collection and processing for the apparatus 500. In one example, the microcontroller 506 can include a hardware multiplication unit, and a computer readable medium containing program code. The microcontroller 506 can also include approximately 8 to 16 ADC inputs capable of converting signals associated with at least one pressure sensor and at least one motion sensor to digital values, and approximately 8 to 16 digital input/output lines capable of monitoring user switches and/or controls. The microcontroller 506 can also include functionality capable of controlling a display driver associated with the output display 508.

In one embodiment, a microcontroller 506 or other processing device can include at least one input and/or output port for transmitting data associated with a pulse of a user or individual. For example, an output port can be a serial output port for downloading or otherwise receiving previously stored pulse data associated with a user or individual. In this example, the serial output port can be connected to a port driver IC (integrated circuit) capable of driving the output port in an associated architecture, such as USB (universal serial bus). The previously stored data can be downloaded from or otherwise received from a data storage device, such as an onboard, non-volatile memory; or on-board or off-board EEPROM (electrically erasable programmable read only memory). In other embodiments, another type of input and/or output port can be utilized with a microcontroller 506 or other processing device in accordance with the invention.

An example of a suitable microcontroller is a dsPIC30F6012 series digital signal controller distributed by Microchip Technology Inc. of Chandler, Ariz. (United States). This type of microcontroller can measure approximately 0.630 by 0.630 by 0.047 inches ( 16 mm by 16 mm by 1.2 mm), and can include a 64 pin package, at least sixteen 12-bit ADCs and 36 additional digital lines, at least two serial communication ports, a 16-bit by 16-bit hardware multiplier, an internal RC (resistive-capacitive) oscillator, and in a low power mode can use approximately 1.5 to 2.0 mA of current when run from 3.3 volts.

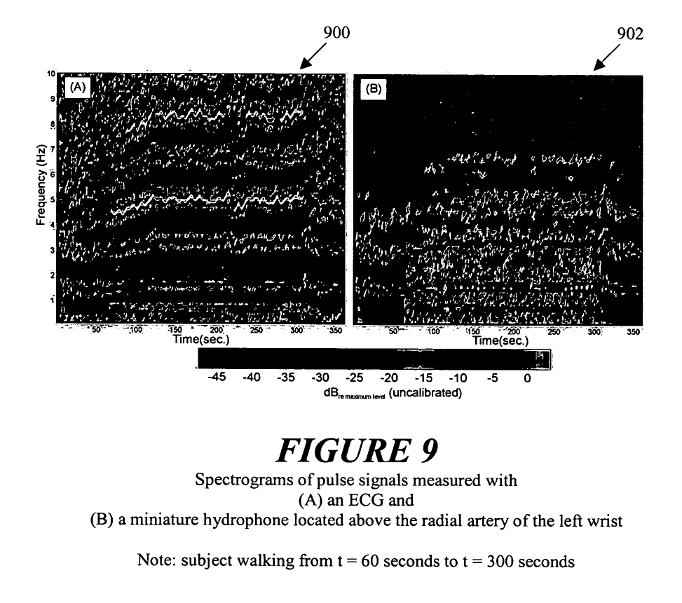

Furthermore, the microcontroller 506 or other processing device can include a computer readable medium containing program code capable of isolating a frequency of a signal associated with a detected pulse of a user or individual. The program code can be further capable of determining frequency components of a measured waveform or signal associated with the detected pulse of the user or individual. In one embodiment, such program code can implement or otherwise utilize an algorithm or technique for processing such signal frequency components, such as a FFT (Fast Fourier Transform) or similar algorithm, technique or method. FIGS. 8 and 9 illustrate examples of measured sensor signals and the implementation of example program code capable of isolating a frequency of a signal or otherwise determining a frequency component of a measured waveform or signal associated with the detected pulse of the user or individual.

FIG. 8 illustrates simultaneously measured pulse time histories by an ECG and by an example sensor of one embodiment of the apparatus 500 in accordance with the invention. Specifically, the upper portion of FIG. 8 shows a time waveform 800 of an ECG signal, and the lower portion shows a time waveform 802 of a non-invasive tonometry-type signal associated with the example sensor. Both signals are shown in 40-second intervals beginning with the subject at rest and then commencing to walk at a steady pace of approximately 2.2 mph (3.7 kph) on a treadmill approximately midway through the record. In one embodiment, program code associated with the microcontroller can implement an algorithm capable of counting a pulse in a measured signal of a sensor. For example, program code can be adapted to evaluate the measured potential of a sensor signal based on predetermined criteria. In one example, three criteria can be evaluated. A first criteria can be whether the measured potential exceeds a predetermined threshold value. Two other criteria can evaluate and measure a local maximum, i.e. higher than either the previous or the subsequent value. If, for example, all three criteria can be met by a particular measured potential, then a pulse beat is determined to exist and can be counted. Other criteria can be implemented by other program code associated with other embodiments of the invention.

FIG. 9 illustrates simultaneously measured pulse time histories by an ECG and by an example sensor of one embodiment of the apparatus 500 in accordance with the invention. Specifically, the left portion of FIG. 9 shows a spectrogram of a measured pulse signal 900 with an ECG, and the right portion shows a spectrogram of a measured pulse signal 902 with the example sensor. Both signals are shown from t=0 to t=360 while the subject was in motion from t=60 seconds to t=300 seconds, shown as spectrograms where the respective instantaneous spectral content is shown as a function of time. The respective pressure signals in each spectrogram contain at least three spectral components relating to the pulse, the fundamental at approximately 1.5 Hz and the first two overtones at approximately 3 Hz and 4.5 Hz.

In other embodiments, other pulse-counting algorithms can be implemented depending on the types of signals received, and the types of devices used to obtain or otherwise detect the pulse of a user or individual. For example, the kurtosis and bandwidth associated with the ECG signal tends to favor time domain-type analysis and associated algorithms. In contrast, the fundamental frequency component of the pressure signal tends to favor frequency domain-type analysis and associated algorithms.

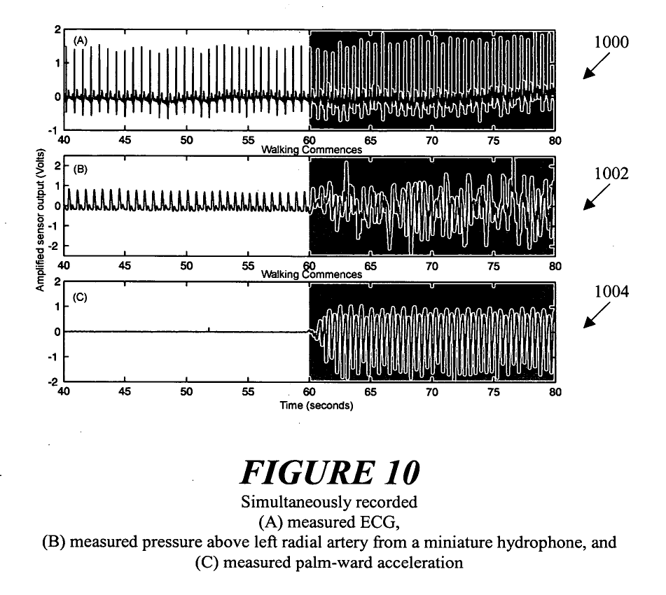

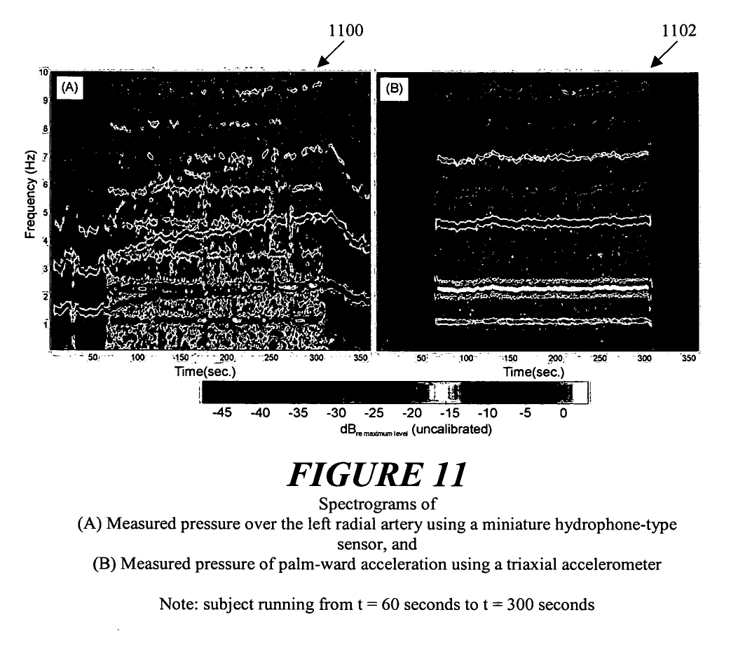

The microcontroller 506 or other processing device can also include a computer readable medium containing program code capable of detecting and measuring a motion or movement of a user or individual, such as motion or movement associated with a user or individual's arm or wrist. In one embodiment, such program code can implement or otherwise utilize an algorithm or technique for processing such signal frequency components, such as a FFT (Fast Fourier Transform) or similar algorithm, technique or method. FIGS. 10 and 11 illustrate examples of measured motion sensor signals and the implementation of example program code capable of detecting and measuring a motion or movement of a user or individual, such as motion or movement associated with a user or individual's arm or wrist.

In one embodiment, the microcontroller 506 can include a computer readable medium containing program code capable of modifying or otherwise correcting a measured pressure signal associated with a detected pulse of a user or individual. In one example, program code can subtract measured or weighted acceleration components from measured or weighted pressure measurements associated with the pulse of a user or individual. In this manner, at least one noise source in tonometric measurements over a user or individual's radial artery, such as the noise produced by arm or wrist motion, can be measured and subtracted from measured or weighted pressure measurements associated with the pulse of a user or individual. In one example, this type of noise can have at least two components, a hydrostatic-type component produced by the change in the height of the measurement point with respect to the heart, and an inertial-type component produced by the acceleration of the measurement point normal to the backplane of the measurement (sensor acceleration) and the acceleration component in the direction of the heart (fluid acceleration). In some instances, activities such as arm waving or running can generate inertial effects which can dominate hydrostatic effects. Some accelerations of interest may have more than one vector component, and at least one algorithm can measure at least three orthogonal acceleration components at a particular location of a pressure sensor or transducer. The algorithm can further determine or otherwise utilize respective weighting coefficients appropriate for each of the three components by numerical minimization of detected energy in the residual signal after the subtraction. This particular algorithm is similar to performing a periodic calibration procedure and, unlike the subtraction itself, may or may not be performed in real time.

Examples of signals processed by an algorithm including a subtraction procedure are shown by FIGS. 10, 11 and 12. In FIG. 10, three examples of signals are shown beginning from the upper portion of the figure and continuing towards the lower portion of the figure. The three types of signals shown correspond to a measured ECG 1000, measured pressure 1002 associated with a user or individual's pulse, and measured acceleration (nominally normal to the pressure sensor's backplane) 1004 associated with a user or individual's motion or movement are shown plotted in a time domain. For the first 60 seconds of the record, shown in the left side portion of the signals in FIG. 10, a user or individual was at rest, and the measured acceleration signal was negligible. At t=60 seconds, shown in the right side portion of the signals in FIG. 10, the user or individual began jogging on a treadmill at approximately 3.2 mph 5.3 kph).

FIG. 11 shows spectrograms for the measured pressure and acceleration signals over approximately six minutes in conditions described in FIG. 10 above. The acceleration signal shown as 1102 on the right side of FIG. 11 comprises a series of overtones of approximately 1.1 Hz, also known as the footfall rate, with the strongest of these overtones measured at approximately 2.2 Hz. The measured pressure signal shown as 1100 on the left side of FIG. 11 comprises similar overtones with the strongest of these overtones also measured at approximately 2.2 Hz. In this example, the measured pulse becomes synchronized with the running motion just as it had been with the previously displayed walking motion in FIG. 10. In other examples, the measured pulse may not be synchronized between different types of motions. For this example, relatively simplified measurement algorithms can be utilized when a user or individual is engaged in harmonic motions such as running, swimming, cycling etc. In some instances, acceleration data can be used as a substitute for a direct measurement of the pulse when noise in the measured pulse signal is relatively high.

An algorithm, such as the algorithm used to process the signals in FIGS. 10 and 11, can utilize one or more weighting coefficients to subtract acceleration from pressure. In one embodiment, one or more weighting coefficients can be determined through minimization of the following equation (1):

$$min {\sum }_{{l}_{1}}^{{l}_{2}} \sqrt{( {P}_{m} ( t ) - {W}_{s} \cdot {a}_{x} ( t ) - {W}_{y} \cdot {a}_{y} ( t ) - {W}_{z} \cdot {a}_{z} ( t ) )} \qquad ( 1 )$$

where Pₘ can represent the measured pressure signal; aₓ, a_{y}, and a_{z} can represent at least three orthogonal components of measured acceleration (x nominally parallels the forearm, y is thumbward when the hand is open, and z is away from the palm); Wₓ, W_{y} and Wₓ can be three real-valued weighting coefficients that are free parameters in the minimization, and t₁ and t₂ can be the time limits over which the minimization is performed.

In some instances, an algorithm using minimization of an equation, such as (1), may reduce the energy in the pulse signal. In some of these instances, if the measured pulse of a user or individual synchronizes to harmonic physical activity, then the measure of activity can be used in the algorithm as an approximate measure of pulse. However, occluding the artery above the pulse measurement point prior to acquiring the data for minimization can improve the calibration procedure by removing the pulse component from the Pₘ used in equation (1).

In other instances, a hydrostatic component of harmonic motion may also be accounted for in the minimization since this component can be 1800 out of phase with the acceleration component. Minimization can produce weighting coefficients for the specific frequency and direction (with respect to the gravity vector) of the recorded arm motion. However, if either of those conditions change, additional techniques can be applied. For instance, the displacement and acceleration have frequency dependencies that differ by a factor of frequency-squared and inverting the forearm can produce a sign flip in the hydrostatic contribution that does not have a corresponding sign flip in the inertial component. One example of an additional technique is by implementing more than one calibration type covering one or more typical activities the user or individual may perform. The algorithm in these instances could be cued to switch calibration coefficients based at least in part on automated identification of associated signatures of each activity type.

At least one suitable implementation of a minimization of equation (1) can be performed using the Nelder-Mead simplex search algorithm implemented with the “fminsearch” m-file in MATLAB®. In this example implementation, four-minute time records sampled at approximately 100 Hz were used for the computation, which took several seconds on a 2.4 GHz personal computer. The implementation could be sped up by reducing the length of the time record, reducing the sampling rate, or seeding the search with previous calibration information if available. As previously mentioned, the operation may or may not be performed in real time. The computational time combined with the length of the time record that is used merely represents a periodic delay in updating the display and imposes a minimum buffer size for the raw input data. The corrected data can be computed from the measured acceleration and pressure using the numerically optimized weighting coefficients described in equation (2) as follows:
P_{c}(t)=Pₘ(t)−Wₓ·aₓ(t)−W_{y}·a_{y}(t)−W_{z}·a_{z}(t) (2)
where P_{c} can be the corrected pressure signal.

FIG. 12 shows an example of a spectrogram of the corrected pressure measurement 1200 resulting from processing the data in FIGS. 10 and 11. The amplitudes of each of the acceleration components have been reduced in comparison to the uncorrected pressure measurement spectrum shown in FIG. 11. The pulse frequency is one dominant feature of this particular spectrogram and can be discerned over the time record shown including the period that it is synchronized with the running motion.

In the example shown, using the corrected or modified signal, a determination of the pulse rate can be made by selecting one or more peak values from a fast Fourier transform (FFT). In this example, a FFT of approximately 10-second records in a first-in-first-out (FIFO) buffer of the time-domain corrected pressure data was relatively effective to provide the data set shown. In this example, a result was obtained that is correct to within approximately 6 beats per minute (bpm), which is the resolution of the chosen transform, over almost the entire data record using an ECG as a baseline.

In some instances, relatively minor data errors may occur, however, such errors are intermittent and have a relatively short duration. In such instances, errors can occur when either an overtone of the pulse or frequency of motion is mistaken for the pulse rate. At least two complementary techniques or error correction algorithms exist to reduce such errors. An example of one complementary technique or error correction algorithm is comparison of the spectral peak with the result of the event-counting algorithm such as the one that was used on an associated ECG signal. Where the former provides a relatively higher result than the latter, it can be replaced with either the result of the counting or the previous value. In addition, the result of the counting algorithm can be used to define the limits of the search for the spectral peak or the previous pulse value can be used to define such limits. For instances where previous values are used for real time computation, the measurement can lock to an overtone of the pulse. In order to test error correction algorithms that may be implemented in real time, substantially longer time records of individual activities can be utilized. Furthermore, errors that may occur in real time can be corrected by smoothing the time history of the recorded pulse after acquiring a relatively long record, such as several minutes or more.

In other instances, some types of motions or movements associated with a user or individual may not be suitable for correction using measured acceleration data or algorithms described above. However, a combination of one or more correction techniques can be implemented for these types of motions or movements. One example of a correction technique can utilize one or more arrays of relatively small sensors capable of differentially filtering out small motions or movements, such as finger tendon motions. Another example of a correction technique can interpolate a user or individual's pulse over a predefined period of time, of which pulse measurements were made before and after the predefined period of time. Yet another example of a correction technique can prompt a user or individual to remain still periodically, or for a predefined period of time, while a pulse measurement is obtained.

There may also be other instances when a particular measured motion or movement in a particular period of time may require correction. In one instance, an impulsive motion of a user or individual's forearm can occur when the individual strikes an object. This may cause a relatively large and brief acceleration signal which may exceed the dynamic range limits of associated amplifiers and analog-to-digital converters used with an apparatus in accordance with the invention. In another instance, a user or individual's arm motion through a relatively dense fluid medium, such as swimming in water, can cause hydrostatic and hydrodynamic pressure contributions to be more significant sources of signal error than inertial effects. Each of these instances, and other similar types of instances, can require a different correction algorithm to be applied to data associated with the measured motion or movement data.

Other corrective techniques or methods can be implemented for handling incorrect pulse rate estimates caused by impulsive-type events or baseline algorithmic errors. If the data is processed for a relatively long time, such as 1-2 minutes, relatively short term heart rate jumps can, in some instances, be rejected if they stray outside a predefined tolerable amount a simple fit (e.g. second order) to the data in that particular time window. Another corrective technique can incorporate any synchronization of pulse rate with motion or movement of a user or individual's body, such as an individual's limb or arm, during certain types of activities, e.g. jogging. If the synchronization is determined to be a common or recurring effect, such phenomena can be utilized to correct pulse rate as needed.

One technique for use with methods, systems, and devices in accordance with embodiments of the invention can enhance noise immunity and identify appropriate strategies for interpolation of a pulse rate during periods when the pulse rate may overwhelmed by noise. Initially, one or more recordings of sensor channels, such as recordings each of 5 sensor channels (ECG, pressure, and three components of acceleration), can be obtained for a user or individual performing a variety of physical activities over a relatively long period of time. From these recording, data can be collected and analyzed to determine and synthesize a variety of sensor channel scenarios. Such data and scenarios can be used to construct a database from which the relative contributions of individual noise sources or components could be characterized and compared to detected or otherwise measured noise components. Comparative and/or pattern recognition-type techniques could then be iteratively applied to define, test, and identify noise sources or components detected or otherwise measured by one or more sensors associated with an apparatus in accordance with the invention.

FIG. 13 illustrates a comparison between various data associated with the pulse of a user or individual. The upper chart 1300 shows data associated with a user or individual who is running. The lower chart 1302 shows data associated with a user or individual who is walking. Both charts compare measured ECG, measured pressures associated with a pulse of a user or individual, modified or corrected pressures associated with a pulse of a user or individual, and peak values from a FFT of pressures associated with a pulse of a user or individual. As shown in this example, the modified or corrected pressure measurements are consistently more accurate than the uncorrected pressure measurements when both are compared to the baseline data of the ECG measurements.

Returning to FIG. 5, the output display 508 shown can include a device capable of providing an output for a user or individual. In one example, the output display 508 can include a custom-type LCD display with a back light. The output display 508 can also include an associated display controller IC (integrated circuit), such as a MAX7234 LCD decoder/driver distributed by Maxim Integrated Products of Sunnyvale, Calif. (United States). Furthermore, the output display 508 can also include a 44-lead package, approximately 0.690 by 0.690 by 0.170 inches (17.5 mm by 17.5 mm by 4.3 mm), and operates on current of approximately 0.05 mA.

The output port 210 shown in FIG. 5 can include a device for transmitting data between the apparatus 500 and a remote device, such as a processing device or a data storage device. In one example, an output port can be USB port. In one embodiment with a USB port, a simple line driver/receiver capable of converting standard TTL digital signal voltages to USB voltage levels can be implemented with the output port. One suitable driver device is the MAX334x series USB transceiver distributed by Maxim Integrated Products of Sunnyvale, Calif. (United States). Such a driver device is approximately 0.250 by 0.200 by 0.043 inches (6.4 mm by 5.1 mm by 1.1 mm). With this type of device, the microcontroller 506 or other processing device may handle some or all of the associated USB software protocol. In another embodiment with a USB port, a full USB controller chip can be implemented with the output port, the USB controller chip capable of handling some or all of the USB protocol including stack storage functionality. With this type of device, the microcontroller 506 or other processing device can transmit instructions or signals through an associated serial port. One suitable USB controller chip is a FT232BM series chip distributed by Future Technology Devices International Ltd. of the United Kingdom. Such a chip is approximately 0.354 by 0.354 by 0.063 inches (9.0 mm by 9.9 mm by 1.6 mm), and can utilize a current of approximately 10 mA or more when operational.

In at least one embodiment, an output port 210 can include a wireless communication device capable of communicating data between the apparatus 104 and a remote device, such as a processing device or data storage device.

In one embodiment, an output port such as 210 can provide a suitable interface with real time, monitoring functions developed with respect to methods, systems, and apparatus for monitoring within-day energy balance deviation, disclosed by U.S. Ser. Nos. 10/903,407, and 60/491,927, previously incorporated by reference. In one example, an associated data acquisition system can be configured to perform real-time processing of sensor data, computation of pulse rate, and energy expenditure. Data can be acquired and processed on a Pentium-4 based personal computer using a PCI-DAS6070 data acquisition board distributed by Measurement Computing Corporation of Middleboro, Mass. (United States). The data acquisition board can be setup and controlled using a graphical user interface (GUI) written and executed, for instance, in MATLAB® or another suitable application program. Data samples can be acquired and displayed for selected input channels, such as a pressure sensor, three motion sensor channels (one for each acceleration axis of the tri-axial sensor), and an ECG signal. The pressure sensor and motion sensor signals can be processed, modified or combined as needed, and plotted on a display screen. An independent, offline calibration and determination of respective motion sensor weighting coefficients, Wₓ, W_{y}, and W_{z}, can be used as inputs to the GUI by the user or individual. Integration of a built-in calibration sequence could be implemented in other embodiments or examples. Additional user inputs to the GUI for signal processing can be sampling frequency, acquisition data block size, and data block size for frequency analysis. Pulse rate can be determined from frequency domain analysis of the processed signal and displayed by the GUI. Energy expenditure can then be computed from the measured pulse rate using a procedure disclosed by U.S. Ser. Nos. 10/903,407, and 60/491,927, and displayed in the GUI as a time history, along with the estimated heart rate. Additionally, heart or pulse rate can be computed from the ECG signal and displayed for the purpose of comparison. User inputs to the GUI for computation of energy expenditure can be gender, age, weight, heart or pulse rate at rest, and an estimated “health index.”

In other embodiments, other algorithms, calculations, deviations and/or health-related balances can be implemented with devices, methods, and systems in accordance with the invention.

The power source 512 shown in FIG. 5 is capable of providing an electrical current to some or all of the other components of the apparatus 500. In one example, a power source 512 can include a lithium coin-type battery. One suitable power source is a 3 volt, lithium, large coin-type battery with a diameter and height of approximately 1 inch by 0.280 inches (25.4 mm by 7.1 mm). Another suitable power source is a ½ AA size lithium battery with a diameter and length of approximately 0.570 inches diameter by 1 inches (14.5 mm by 25.4 mm).

In one embodiment, a power source can also include a switching voltage regulator capable of maintaining a predetermined voltage level when battery voltage drops or the batter power is otherwise consumed. For example, in one embodiment with a 3 volt lithium coin-type battery, as battery power is consumed, the battery voltage may drop to approximately 2 volts. A boost-type switching voltage regulator can be utilized with the power source to maintain the voltage at approximately 3 volts. One suitable switching voltage regulator is a MAX683x series, low power step up switching voltage regulator distributed by Maxim Integrated Products of Sunnyvale, Calif. (United States).

In other embodiments of an apparatus shown in FIG. 5, other electrical or mechanical-type components can be utilized. For example, other components can include, but are not limited to, a printed circuit board, one or more switches for a user interface, a USB port connector, a power source or battery holder, a power source or battery charging connector, one or more resistors, one or more capacitors, or a switching voltage regulator inductor. One skilled in the art will recognize the applicability of these and other components with other embodiments in accordance with the invention.

For example, an apparatus such as the wrist worn device 104 can provide information related to a person's energy expenditure, such as a pulse rate over a period of time, to an algorithm capable of calculating an energy balance function based in part on an energy expenditure and energy intake over a period of time. The wrist worn device 104 could then determine, output, or otherwise display information associated with the energy balance function.

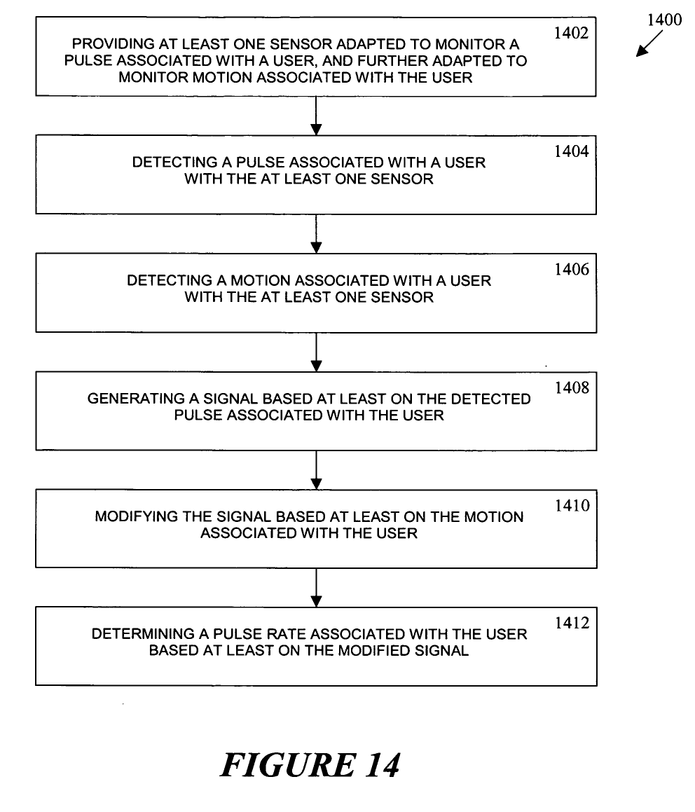

FIG. 14 illustrates a flowchart of a method 1400 of measuring a pulse of an individual according to an embodiment of the present invention. The method 1400 can be implemented by a system or apparatus, such as the apparatus 104 in FIGS. 1-4, or the apparatus 500 in FIG. 5.

The method begins at block 1402. At block 1402, at least one sensor adapted to monitor a pulse associated with a user, and further adapted to monitor motion associated with the user is provided. In at least one embodiment, the at least one sensor can be mounted to the user on at least one of the following locations: arm, leg, head, neck, chest, calf, ankle, wrist, finger, hand, foot, toe, or a body part. In another embodiment, the at least one sensor can be mounted to the user on at least one of the following locations: arm, leg, head, neck, chest, calf, ankle, wrist, finger, hand, foot, toe, or a body part. In yet another embodiment, the at least one sensor can be mounted to at least one of the following: a wrist-worn device, a casing, a patch, a band, or an article of clothing. Furthermore, the at least one sensor comprises at least one of the following: a piezoelectric sensor, a force transducer, a pressure transducer, an electret foam sensor, a pressure sensor, a non-invasive tonometric sensor, a motion sensor, an accelerometer, or an array of pressure sensors and motion sensors.

Block 1402 is followed by block 1404, in which a pulse associated with a user is detected with the at least one sensor.

Block 1404 is followed by block 1406, in which motion associated with a user is detected with the at least one sensor.

Block 1406 is followed by block 1408, in which a signal based at least on the detected pulse associated with the user is generated.

Block 1408 is followed by block 1410, in which the signal is modified based at least on the motion associated with the user.

Block 1410 is followed by block 1412, in which a pulse rate associated with the user is determined based at least on the modified signal.

In another embodiment, additional elements of method 1400 can exist, such as calculating an energy balance based at least on the pulse rate, and outputting an energy balance calculation to the user.

In yet another embodiment, an additional element of method 1400 can exist, such as transmitting the pulse rate to a processing device adapted to store the pulse rate.

In block 1412, the method 1400 ends.

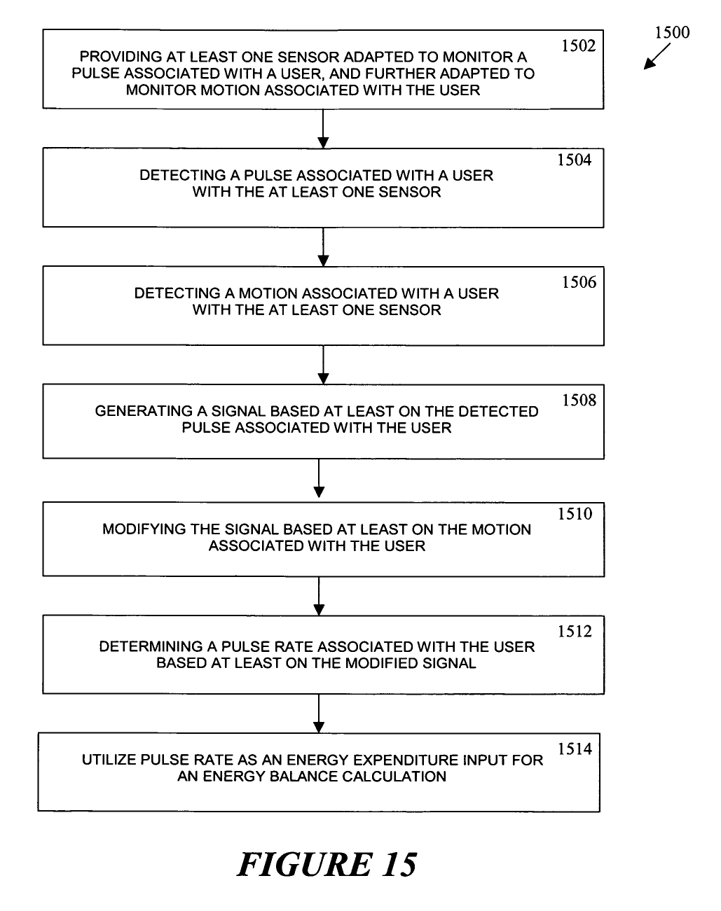

FIG. 15 illustrates a flowchart of a method 1500 of measuring a pulse of an individual as an energy expenditure input to an energy balance calculation according to an embodiment of the present invention. The method 1500 can be implemented by a system or apparatus, such as the apparatus 104 in FIGS. 1-4, or the apparatus 500 in FIG. 5.

The method begins at block 1502. At block 1502, at least one sensor adapted to monitor a pulse associated with a user, and further adapted to monitor motion associated with the user is provided. In at least one embodiment, the at least one sensor can be mounted to the user on at least one of the following locations: arm, leg, head, neck, chest, calf, ankle, wrist, finger, hand, foot, toe, or a body part. In another embodiment, the at least one sensor can be mounted to the user on at least one of the following locations: arm, leg, head, neck, chest, calf, ankle, wrist, finger, hand, foot, toe, or a body part. In yet another embodiment, the at least one sensor can be mounted to at least one of the following: a wrist-worn device, a casing, a patch, a band, or an article of clothing. Furthermore, the at least one sensor comprises at least one of the following: a piezoelectric sensor, a force transducer, a pressure transducer, an electret foam sensor, a pressure sensor, a non-invasive tonometric sensor, a motion sensor, an accelerometer, or an array of pressure sensors and motion sensors.

Block 1502 is followed by block 1504, in which a pulse associated with a user is detected with the at least one sensor.

Block 1504 is followed by block 1506, in which motion associated with a user is detected with the at least one sensor.

Block 1506 is followed by block 1508, in which a signal based at least on the detected pulse associated with the user is generated.

Block 1508 is followed by block 1510, in which the signal is modified based at least on the motion associated with the user.

Block 1510 is followed by block 1512, in which a pulse rate associated with the user is determined based at least on the modified signal.

In another embodiment, additional elements of method 1500 can exist, such as calculating an energy balance based at least on the pulse rate, and outputting an energy balance calculation to the user.

In yet another embodiment, an additional element of method 1500 can exist, such as transmitting the pulse rate to a processing device adapted to store the pulse rate.

Block 1512 is followed by block 1514, in which the pulse rate is utilized as an energy expenditure input for an energy balance calculation. In the example shown, the energy expenditure input can be a value or measurement for a heart rate, pulse rate, or any other quantitative input to an energy balance calculation or routine. One example of a suitable energy balance calculation or routine is associated with, a “Health Watch,” as disclosed by U.S. Ser. No. 10/903,407, and U.S. Ser. No. 60/491,927, wherein the contents of both applications have previously been incorporated by reference.

In block 1514, the method 1500 ends.

Changes and modifications, additions and deletions may be made to the structures and methods recited above and shown in the drawings without departing from the scope of the invention and the following claims.

The invention we claim is:

1. A method for measuring a pulse rate of an individual, the method characterized by:
providing at least one sensor adapted to monitor a pulse associated with a user, and further adapted to monitor motion associated with the user;
detecting a pulse associated with a user with the at least one sensor;
detecting motion associated with a user with the at least one sensor;
generating a signal based at least on the detected pulse associated with the user;
modifying the signal based at least on the motion associated with the user; and
determining a pulse rate associated with the user based at least on the modified signal.

2. The method of claim 1, is further characterized by:
calculating an energy balance based at least on the pulse rate; and
outputting an energy balance calculation to the user.

3. The method of claim 1, further characterized by:
transmitting the pulse rate to a processing device adapted to store the pulse rate.

4. The method of claim 1, wherein the at least one sensor can be mounted to the user on at least one of the following locations: arm, leg, head, neck, chest, calf, ankle, wrist, finger, hand, foot, toe, or a body part.

5. The method of claim 1, wherein the at least one sensor can be mounted to at least one of the following: a wrist-worn device, a casing, a patch, a band, or an article of clothing.

6. The method of claim 1, wherein the at least one sensor is further characterized by at least one of the following: a piezoelectric sensor, a force transducer, a pressure transducer, an electret foam sensor, a pressure sensor, a non-invasive tonometric sensor, a motion sensor, an accelerometer, or an array of pressure sensors and motion sensors.

7. An apparatus for measuring pulse rate of an individual, the apparatus characterized by:
at least one sensor adapted to:
detect a pulse associated with a user with the at least one sensor;
detect motion associated with a user with the at least one sensor;
generate a signal based at least on the detected pulse associated with the user; and
a processor adapted to:
receive the signal from the at least one sensor;
modify the signal based on at least the motion associated with the user; and determine a pulse rate associated with the user based at least on the modified signal.

8. The apparatus of claim 7, further comprising:
an output device adapted to display the pulse rate.

9. The apparatus of claim 7, wherein the processor is further adapted to:
calculate an energy balance based at least on the pulse rate; and
output an energy balance calculation to the user.

10. The apparatus of claim 7, wherein the processor is further adapted to:
transmit the pulse rate to a processing device adapted to store the pulse rate.

11. The apparatus of claim 7, wherein the at least one sensor can be mounted to the user on at least one of the following locations: arm, leg, head, neck, chest, calf, ankle, wrist, finger, hand, foot, toe, or a body part.

12. The apparatus of claim 7, wherein the at least one sensor is mounted to at least one of the following: a wrist-worn device, a casing, a patch, a band, or an article of clothing.

13. The apparatus of claim 7, wherein the at least one sensor is further characterized by at least one of the following: a piezoelectric sensor, a force transducer, a pressure transducer, an electret foam sensor, a pressure sensor, a non-invasive tonometric sensor, a motion sensor, an accelerometer, or an array of pressure sensors and motion sensors.

14. A system for measuring a pulse rate of an individual and determining the individual's energy expenditure while the individual's body is in motion, the system characterized by: at least one sensor array capable of:
detecting a pulse associated with a user with the at least one sensor array;
detecting motion associated with a user with the at least one sensor array;
generating a signal based at least on the detected pulse associated with the user; and
a processor capable of:
receiving the signal from the at least one sensor array;
modifying the signal based on at least the motion associated with the user;
determining a pulse rate associated with the user based at least on the modified signal;
calculating an energy balance based at least on the pulse rate; and
outputting an energy balance calculation to the user.

15. The system of claim 14, further characterized by:
an output device capable of:
displaying the pulse rate;
displaying the energy balance calculation.

16. The system of claim 14, wherein the processor is further capable of:
transmitting the pulse rate and the energy balance to a data storage device capable of storing the pulse rate and the energy balance.

17. The system of claim 14, wherein the at least one sensor array can be mounted to the user on at least one of the following locations: arm, leg, head, neck, chest, calf, ankle, wrist, finger, hand, foot, toe, or a body part.

18. The system of claim 14, wherein the at least one sensor array can be mounted to at least one of the following: a wrist-worn device, a casing, a patch, a band, or an article of clothing.

19. The system of claim 14, wherein the at least one sensor array is further characterized by at least one of the following: a piezoelectric sensor, a force transducer, a pressure transducer, an electret foam sensor, a pressure sensor, a non-invasive tonometric sensor, a motion sensor, an accelerometer, or an array of pressure sensors and motion sensors.

20. The system of claim 14, wherein the at least one sensor array is further characterized by at least one pressure sensor and at least one motion sensor.
